(19) United States
(12) Patent Application Publication
(10) Pub. No.: US 2006/0198836 A1
(43) Pub. Date: Sep. 7, 2006

(54) Method of treating hit patients with argatroban

(75) Inventors: Margaret M. Gardner, King of Prussia, PA (US); Marcie J. Hursting, King of Prussia, PA (US); Elizabeth A. Tarka, King of Prussia, PA (US); Catherine N. Verme-Gibboney, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: Smithkline Beecham Corporation, Philiadelphia, PA (US)

(21) Appl. No.: 10/564,947

(22) PCT Filed: Jul. 16, 2004
(86) PCT No.: PCT/US04/22946

§ 371 Date: Jan. 17, 2006

Related U.S. Application Data
(60) Provisional application No. 60/488,056, filed on Jul. 17, 2003.

(51) Int. Cl. A61K 38/48

(52) U.S. Cl. 424/94.64

(57) ABSTRACT

This invention relates to the improved methods of use of Argatroban in heparin-induced thombocytopenia.

### FIELD OF THE INVENTION

The present invention discloses improved methods of treating patients suffering from heparin-induced thrombocytopenia.

### BACKGROUND OF THE INVENTION

Heparin-induced thrombocytopenia (HIT) is a devastating, immune-mediated complication of heparin therapy that can lead to serious arterial and venous thrombosis with the untoward consequences of limb amputation and even death. See Deitcher S R, Carman T L. Heparin-induced thrombocytopenia: natural history, diagnosis, and management. Vascular Med 2001;6:113-119. Hirsh J, Warkentin T E, Shaughnessy S G, et al. Heparin and low-molecular-weight heparin: mechanisms of action, pharmacokinetics, dosing, monitoring, efficacy, and safety. Chest 2001; 119:64S-94S. Lewis B E, Wallis D E, Berkowitz S D, et al. Argatroban anticoagulant therapy in patients with heparin-induced thrombocytopenia. Circulation. 2001;103:1838-1843, incorporated in their entirety by reference. HIT typically onsets 5 to 15 days after starting heparin therapy (or earlier if the patient has been previously exposed to heparin) and may occur with any heparin given at any dose and by any route. Kelton J G. Argatroban—a novel thrombin-specific inhibitor for the treatment of heparin-induced thrombocytopenia. Today's Therap Trends 2002; 20:15-35, incorporated in its entirety by reference.

The diagnosis is usually made on a clinical basis with the development of thrombocytopenia (ie, defined as a platelet count <100-150×10⁹/L, or a 50% decrease in the platelet count from baseline) and/or the development of new thrombosis following heparin exposure. For treating patients with HIT, all sources of heparin must be eliminated. Because of the highly prothrombotic nature of HIT, and because 50% of patients managed by heparin cessation alone experience thrombosis following discontinuation of heparin, Warkentin T E, Kelton J G. A 14-year study of heparin-induced thrombocytopenia. Am J Med. 1996;101:502-507, incorporated in its entirety by reference, the initiation of an alternative parenteral anticoagulant such as a direct thrombin inhibitor (DTI), for example Argatroban, is recommended for appropriate therapy. Deitcher S R, Carman T L. Heparin-induced thrombocytopenia: natural history, diagnosis, and management. Vascular Med 2001;6:113-119. Hirsh J, Warkentin T E, Shaughnessy S G, et al. Heparin and low-molecular-weight heparin: mechanisms of action, pharmacokinetics, dosing, monitoring, efficacy, and safety. Chest 2001; 119:64S-94S, incorporated in their entirety by reference. According to HIT treatment guidelines, Hirsh J, Warkentin T E, Shaughnessy S G, et al. Heparin and low-molecular-weight heparin: mechanisms of action, pharmacokinetics, dosing, monitoring, efficacy, and safety. Chest 2001; 119:64S-94S, incorporated in their entirety by reference, alternative anticoagulation should be continued in patients with acute HIT with or without thrombosis. These same guidelines recommend against using coumarin derivatives as sole therapy in acute HIT because of the risk of causing venous limb gangrene, Hirsh J, Warkentin T E, Shaughnessy S G, et al. Heparin and low-molecular-weight heparin: mechanisms of action, pharmacokinetics, dosing, monitoring, efficacy, and safety. Chest 2001; 119:64S-94S, incorporated in their entirety by reference, which can lead to amputation. Warkentin T E, Elavathil L J, Hayward C P M, et al. The pathogenesis of venous limb gangrene associated with heparin-induced thrombocytopenia. Ann Intern Med. 1997;127:804-812, incorporated in their entirety by reference.

In the United States, Argatroban is indicated as an anticoagulant for prophylaxis or treatment of thrombosis in patients with HIT and as an anticoagulant in patients with or at risk for HIT undergoing percutaneous coronary interventions (effective April, 2002). There is only one other agent (lepirudin) in the United States that is FDA-approved for the treatment of thrombosis associated with HIT.

As stated in the prescribing information for Argatroban, the recommended initial dose for prophylaxis or treatment of thrombosis in adult patients with HIT is 2 mcg/kg/min (reduced to 0.5 mcg/kg/min in patients with moderate hepatic impairment). The dose is adjusted, not to exceed 10 mcg/kg/min, to achieve a steady-state activated partial thromboplastin time (aPTT) that is 1.5 to 3 times the baseline aPTT value, not to exceed 100 seconds. The prescribing information also gives general guidance on the conversion to oral anticoagulant therapy (warfarin), including describing the effects of Argatroban and warfarin cotherapy on the international normalized ratio (INR), recommending against use of a loading dose of warfarin, and recommending that warfarin therapy be initiated with the expected daily dose. Patients receiving parenteral anticoagulation for any medical condition, including HIT, may be converted to oral therapy (such as warfarin) for long-term oral anticoagulation for various underlying medical conditions. Following initiation of warfarin, a patient's vitamin K-dependent anticoagulant proteins C and S decline rapidly, resulting in decreased inhibition of the procoagulant factors VIIIa and VIIa, an upsurge in thrombin generation, and a transient hypercoagulable condition. Stirling Y. Warfarin-induced changes in procoagulant and anticoagulant proteins. Blood Coagul Fibrinolysis. 1995;6:361-73, incorporated in their entirety by reference.

Patients who have HIT are already profoundly hypercoagulable due to the syndrome, and thus are at an increased risk of thrombotic complications during this time if coumarin derivatives are initiated too early (i.e. before the platelet count has fully recovered). It has previously been hypothesized that venous limb gangrene during initiation of warfarin therapy is due to a combination of factors, including an initial thrombotic event requiring heparin anticoagulation, followed by a prothrombotic state such as HIT, and high doses of warfarin (such as 15-20 mg daily). In fact, cases have been reported of venous limb gangrene occurring during overlapping therapy of a DTI and warfarin when the DTI was interrupted during persisting thrombocytopenia (which would indicate a continued hypercoagulable state). Stirling Y. Warfarin-induced changes in procoagulant and anticoagulant proteins. Blood Coagul Fibrinolysis. 1995;6:361-73. Smythe M A, Warkentin T E, Stephens J L, et al. Venous limb gangrene during overlapping therapy with warfarin and a direct thrombin inhibitor for immune heparin-induced thrombocytopenia. Am J Hematol 2002;71:50-52, incorporated in their entirety by reference.

Recent exploratory analyses of the relationships between patient outcomes, dosing and platelet counts in the Argatroban pivotal studies have provided insights into approaches for refining the existing method of therapy in HIT patients using Argatroban.

### SUMMARY OF THE INVENTION

The present invention provides specific treatment protocols for administering anticoagulants, especially Argatroban, involving continuous administration of the anticoagulant until platelet counts have substantially recovered. The present methods are also applicable to methods of treatment to convert Argatroban to oral anticoagulation such as warfarin when such conversion is warranted.

### DETAILED DESCRIPTION OF THE INVENTION

On the basis of advances in the understandings of HIT and its pathogenesis, the current recommendations for safer treatment approaches, and insights from our recent analyses of the pivotal studies of Argatroban in HIT, the present invention involves modification of the Argatroban prescribing information to describe a “specific” method of treatment. The specific method involves the continuation of Argatroban therapy until platelet counts have recovered substantially. The specificity of continuing anticoagulation until platelet counts recover to >100×10⁹/L or returned to pre-heparin induced thrombocytopenia platelet baseline count for those patients whose platelet counts demonstrated a 50% drop from baseline ensures continuous anticoagulation to avoid further prothrombotic effects resulting from the profound hypercoaguable syndrome of HIT and a potential premature discontinuation of Argatroban. This specific method of treatment is also applicable in the method of treatment to convert Argatroban to oral anticoagulants (coumarin derivatives such as warfarin), when oral anticoagulation is needed.

The specific method of treatment is such that, when conversion to oral anticoagulation is needed, coumarin derivatives should not be initiated until substantial recovery of the platelet count has been achieved during Argatroban therapy. In addition, the overlap of Argatroban and warfarin therapy should be continued for 4 or 5 days duration, to allow enough time to inhibit the vitamin-K-dependent clotting factors. This specific methodology will assist healthcare professionals to not only ensure continuous anticoagulation and avoid prothrombotic effects resulting from the underlying HIT syndrome but also minimize the risk of warfarin-induced venous gangrene. The potential adverse events resulting from a premature discontinuation of a DTI (e.g., Argatroban), premature initiation of coumarin derivatives in HIT patients, and/or inadequate duration of combined Argatroban and warfarin therapies in HIT patients can be minimized by this method of treatment. This specific method of treatment improves outcomes in patients with HIT and promotes the safe conversion from Argatroban to warfarin, when needed.

As used herein “treatment” of a patient includes, but is not limited to prophylaxis of thrombosis in isolated heparin-induced thrombocytopenia.

As used herein “isolated heparin-induced thrombocytopenia” implies a HIT patient not suffering from thrombosis related to HIT.

As used herein “substantial recovery of platelet counts” implies that platelet counts have recovered to >100,000×10⁹/L or returned to pre-heparin induced thrombocytopenia platelet baseline count for those patients whose platelet counts demonstrated a 50% drop from baseline.

In addition to Argatroban, lepirudin (Refludan) is an approved HIT agent for patients with HIT and associated thromboembolic disease. The recommended dosage as per Refludan's PI is an initial intravenous bolus dose, followed by continuous infusion for 2 to 10 days or longer if clinically needed. The method of use for Refludan in switching to oral anticoagulation is as follows:

“If a patient is scheduled to receive coumarin derivatives (vitamin K antagonists) for oral anticoagulation after Refludan therapy, the dose of Refludan should first be gradually reduced in order to reach an aPTT ratio just above 1.5 before initiating oral anticoagulation. Coumarin derivatives should be initiated only when platelet counts are normalizing. The intended maintenance dose should be started with no loading dose. To avoid prothrombotic effects when initiating coumarin, continue parenteral anticoagulation for 4 to 5 days (see oral anticoagulant package insert for information). The parenteral agent can be discontinued when the INR stabilizes within the desired target range.”

Other anticoagulants pertinent to the present invention include:
Lovenox (enoxaparin)—This is a low molecular weight heparin and is contraindicated as an anticoagulant in patients with HIT;
Fragmin (dalteparin)—This is a low molecular weight heparin and, as such, contraindicated as an anticoagulant for patients with HIT;
Innohep (tinzaparin)—This is a low molecular weight heparin and, as such, contraindicated as an anticoagulant for patients with HIT;
Angiomax (bivalirudin)—This is a DTI. There is limited information for the use in patients with HIT. As it does not have an FDA indication for HIT, there is insufficient clinical data to support a method of treatment;
Arixtra (fondaparinux)—This is a Factor Xa inhibitor and is not indicated for heparin-induced thrombocytopenia. Again there is insufficient clinical data;
Refludan (lepirudin)—This is a specific DTI. It is recombinant hirudin derived from yeast cells. For the indication and method, please see the note above; and
Danaparoid (low molecular weight heparin)—This was discontinued in the U.S. Market.

There are several parenteral anticoagulants which are DTIs that are currently on the market or have been evaluated in major clinical trials (e.g., Argatroban, bivalirudin, efegatran, inogatran, desirudin, and lepirudin). Only Argatroban, bivalirudin, lepirudin and desirudin are approved and in the US Market and only Argatroban and lepirudin are approved for use in HIT patients.

As used herein “thromboembolic complications” include, but are not limited to: pulmonary embolism, stroke or cerebral thrombosis, any deep vein thrombosis (distal, proximal, and bilateral), myocardial infarction, arterial occlusion and thrombosis.

In a clinical trial of 304 patients with HIT with or without thrombosis who received argatroban, 165 patients with active HIT had platelet count data available for the day of argatroban cessation. Of these patients, 41 patients had a platelet count <100×10⁹/L and 124 patients had a platelet count of >100×10⁹/L on the day that argatroban therapy was discontinued. In the group with lower platelet counts, 61% of patients experienced the composite endpoint of either death, amputation or new thrombosis, compared to 31.5% in the group that had higher platelet counts on the day argatroban was discontinued. Thus, these data provide additional evidence that premature discontinuation of a DTI is detrimental in patients with HIT. These data collectively demonstrate a significant safety issue in the treatment of patients with HIT that is not adequately addressed in the current labeling for Argatroban. Therefore, we recommend these approaches for refining the existing method of therapy in patients with HIT receiving Argatroban.

The following examples are intended to illustrate the present invention and not be limiting in any way:

### EXAMPLE 1

### Patient Case—Argatroban Monotherapy (No Conversion to Warfarin)

PC is a 63 year-old man with a history of hypertension and hyperlipidemia who presented to the emergency department (ED) complaining of chest pain. His EKG revealed 5-6 mm ST depression in leads II, III, and aVF. The patient was admitted to the Coronary Care Unit and was treated with aspirin, intravenous nitroglycerin, metoprolol, and intravenous heparin. The platelet count on admission was 212×10⁹/L. Serial cardiac enzymes and troponin concentrations were negative for a myocardial infarction. However, because of continued chest pain, the patient underwent cardiac catheterization, which revealed severe 3-vessel coronary artery disease. Coronary artery bypass grafting was performed 2 days later with an uneventful postoperative course. He was discharged from the hospital 7 days after the surgery in good condition and his platelet count was 136×10⁹/L. One week later, the patient returned to the ED complaining of the acute onset of a cold and painful swollen left leg. His medications included aspirin 325 mg daily, metoprolol 75 mg twice daily, NitroDur 20 mg daily and atorvastatin 20 mg daily. The physical examination was normal with the exception of mild hypertension and a cold, swollen left leg with blue discoloration from the knee down. In addition, the left popliteal pulse was faint and the left ankle pulse was non-palpable. Routine laboratory studies were normal except for a platelet count of 41×10⁹/L.

A left femoral arteriogram revealed extensive atherosclerotic disease of the superficial femoral artery and occlusion of the popliteal artery. Intravenous heparin was started and the patient was sent to the operating room for emergency surgery. A femoro-popliteal bypass was performed with a graft and there was an initial return of arterial blood flow to the lower leg. However, before the incision was closed, the graft re-occluded. The graft was opened and explored with an embolectomy catheter. A large amount of whitish granular material was found and subsequently removed from the distal end of the graft. Pulses returned to the lower leg and the incision was closed. By the time the closure was complete, the graft had again re-occluded. The finding of a platelet-rich “white clot” during this surgery along with thrombocytopenia alerted physicians to suspect HIT with thrombosis (HITT).

Heparin was immediately discontinued and the DTI Argatroban was initiated (at 2 mcg/kg/min) that evening (day 1 of Argatroban). On the following day, the patient's left lower leg remained cold, blue and pulseless. He was transferred to another hospital where an amputation was performed. Argatroban was continued for 10 days. The platelet count began improving with the following serial platelet counts: 62×10⁹/L on day 4, 78×10⁹/L on day 6, 110×10⁹/L on day 8 and 180×10⁹/L on day 10. The patient was measured for a prosthetic leg and he underwent intense physical therapy for 6 months.

### EXAMPLE 2

### HIT Treated with Argatroban and Conversion to Warfarin

A 32-year old female experienced pregnancy-induced hypertension and sudden weight gain during the third trimester of pregnancy. Premature labor led to the delivery of twins at 31 weeks gestation. After delivery, the patient remained hypertensive. On post-partum day 7, she awoke with chest pain and shortness of breath. She was lethargic with a blood pressure of 190/96 mm Hg and heart rate 135 beats per minute. Chest X-ray demonstrated mild pulmonary congestion, and electrocardiogram indicated sinus tachycardia but was otherwise normal. The platelet count was 406×10⁹/L. A heparin infusion was initiated and the patient underwent a ventilation/perfusion scan, which revealed intermediate probability for diagnosis of pulmonary embolism.

On postpartum day 10, the patient was transferred to another hospital for left hemiparesis. A non-hemorrhagic right cerebral infarction was confirmed by computed tomography. During days 4 to 7 of heparin therapy, platelet counts fluctuated between 163×10⁹/L and 99×10⁹/L with no clear downward trend. A platelet count was not obtained on day 8. However, on day 9 of heparin therapy, the patient's right leg became ischemic and her platelet count decreased to 34×10⁹/L. HIT was suspected and heparin was immediately discontinued. The patient was taken to the operating room and Fogerty balloon thrombectomy of the right superficial and profunda arteries removed a typical red, fleshy acute thrombus. The clinical diagnosis of HIT with thrombosis was confirmed by the detection of heparin/PF4 antibodies in the patient's day 9 serum (but not in her serum from day 3) using an in-vitro functional test, the serotonin release assay (SRA).

An alternative anticoagulant was needed for further treatment of her pulmonary embolism and to prevent further thrombosis of the right femoral artery. The direct thrombin inhibitor Argatroban was initiated at 2 mcg/kg/min and titrated to achieve an activated partial thromboplastin time (aPTT) of 1.5 to 3.0 times the patient's baseline aPTT value. Over the next 6 days during Argatroban infusion, the patient's platelet count recovered rapidly and there were no further thromboembolic events or bleeding complications.

| Hospital Day | Day of Argatroban Therapy | Platelet Count |
|---|---|---|
| 9 | Prior to initiation | 34 × 10⁹/L |
| 10 | 2 | 55 × 10⁹/L |
| 13 | 5 | 110 × 10⁹/L |
| 14 | 6 | 160 × 10⁹/L |
| 16 | 8 | 149 × 10⁹/L |
| 18 | 10 | 210 × 10⁹/L |
| 20 | 12 | 280 × 10⁹/L |

After 6 days of Argatroban monotherapy and recovery of the platelet count to >100×10⁹/L, warfarin was initiated concomitantly. Warfarin was started at the expected daily dose of 5 mg. On the 4^{th} day of concomitant therapy, Argatroban infusion was held for 4 hours (to eliminate the effects of Argatroban on the INR) and the INR was rechecked to assure a therapeutic INR on warfarin alone. The INR was 2.1, which is considered therapeutic for the underlying medical condition, so the Argatroban was discontinued.

The patient's pulmonary function continued to improve, and she regained a significant amount of motor function. She was able to ambulate with a walker, and was discharged from the hospital on day 25. The platelet count on discharge was 410×10⁹/L.

### EXAMPLE 3

### Patient Case—Avoiding Warfarin-induced Skin Necrosis and Venous Limb Gangrene

Several recent reports illustrate the importance of an appropriate method for transitioning a patient with HIT from a direct thrombin inhibitor to coumarin derivatives. The great majority of patients with HIT will require treatment with oral anticoagulants either for the initial thromboembolic event, for thromboses that arose secondary to HIT, or for protection from the extreme risk of new thrombosis in isolated HIT (without thrombosis at diagnosis of HIT).

The following patient case is an example of an appropriate transition to warfarin, because Argatroban therapy is continued for at least 5 days and until the patient's platelet count has recovered to >100×10⁹/L. In addition, concomitant therapy with Argatroban and warfarin overlaps for at least 4-5 days.

A 72 year old female presented to the hospital with an acute myocardial infarction, for which she received tissue plasminogen activator and heparin. Her platelet count was 190×10⁹/L on admission. She underwent coronary artery bypass grafting several days later, and heparin was continued throughout the peri-operative period. Postoperatively, the patient experienced atrial fibrillation, and warfarin was initiated. Four days postoperatively, she developed pain and discoloration of the left breast and right leg. The platelet count was 60×10⁹/L and the INR was 6.1. Unfortunately, the breast and leg lesions progressed rapidly and the patient ultimately required a mastectomy and below the knee amputation. Five weeks later, she was readmitted with upper extremity swelling. The platelet count was 126×10⁹/L on readmission. Ultrasound confirmed axillary/subclavian and femoral venous thromboses, for which heparin therapy was re-initiated. The platelet count fell from 126×10⁹/L to 26×10⁹/L after 3 days of intravenous heparin, and the diagnosis of HIT with thrombosis was made. The patient's initial breast and leg lesions are examples of warfarin-induced skin necrosis and venous limb gangrene, respectively, which are possible clinical manifestations of HIT. The patient was started on Argatroban 2 mcg/kg/min and the dose was titrated to maintain aPTT values between 1.5 and 3 times her baseline aPTT. An inferior vena cava filter was placed. Argatroban was continued for 11 days. On day 6 of Argatroban therapy, warfarin was initiated at the estimated daily dose of 5 mg. At that time, the platelet count was 150×10⁹/L. Concomitant therapy with Argatroban and warfarin was continued for 4 days in accordance with the recommended overlap of 4 to 5 days. On the 5^{th} day of concomitant therapy, the Argatroban was held for 6 hours and the INR value reflecting warfarin therapy alone was checked. The INR was 2.9 and the Argatroban infusion was subsequently discontinued.
See e.g.:
Warkentin T E, Elavathil L J, Hayward C P M, et al. The pathogenesis of venous limb gangrene associated with heparin-induced thrombocytopenia. Ann Intern Med. 1997; 127:804-812.
Smythe M A, Warkentin T E, Stephens J L, et al. Venous limb gangrene during overlapping therapy with warfarin and a direct thrombin inhibitor for immune heparin-induced thrombocytopenia. Am J Hematol 2002; 71:50-52.
Srinivasan A F, Rice L, Bartholomew J R, et al. Warfarin-induced skin necrosis and venous limb gangrene in the setting of heparin-induced thrombocytopenia. Arch Int Med 2004;164:66-70.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore the examples herein are to be construed as merely illustrative and not a limitation of the scope of the present invention in any way. The embodiments of the invention in which an exclusive property or privilege is claimed are defined as follows:

What is claimed is:

1. A method of treating patients with or at risk for heparin-induced thrombocytopenia, including the prophylaxis and treatment of thrombosis in heparin-induced thrombocytopenia, comprising continually administering to a patient with or at risk for HIT, with or without thrombosis, an anticoagulant until platelet counts in said patient have recovered substantially.

2. A method according to claim 1 wherein the anticoagulant is selected from the group consisting of lepirudin, enoxaparin, dalteparin, tinzaparin, bivalirudin, and fondaparinux.

3. A method according to claim 1 wherein the anticoagulant is a direct thrombin inhibitor.

4. A method according to claim 3 wherein the direct thrombin inhibitor is selected from the group consisting of Argatroban, bivalirudin, efegatran, inogatran, desirudin, and lepirudin.

5. A method according to claim 4 wherein the direct thrombin inhibitor is Argatroban or lepirudin

6. A method according to claim 5 wherein the direct thrombin inhibitor is Argatroban.

7. A method of preventing an upsurge in thrombin regeneration and the prevention of new thromboembolic complications in patients with or at risk for HIT, with or without thrombosis, comprising continually administering to a patient with or at risk for HIT, with or without thrombosis, an anticoagulant until platelet counts in said patient have recovered substantially.

8. A method of lowering the risk of venous limb gangrene in a patient in need of HIT treatment comprising continually administering to a patient with or at risk for HIT, with or without thrombosis, an anticoagulant until platelet counts in said patient have recovered substantially.

9. A method of lowering the risk of amputation in a patient in need of HIT treatment comprising continually administering to a patient with or at risk for HIT, with or without thrombosis, an anticoagulant until platelet counts in said patient have recovered substantially.

10. A method of treating HIT involving the co-administration of Argatroban and a coumarin derivative comprising the administration of Argatroban until substantial recovery of platelet count, followed by the administration of the coumarin derivative, such that there is overlap in Argatroban and coumarin derivative therapy.

11. A method according to claim 9 wherein the coumarin derivative is warfarin.

12. A method according to claim 10 such that the overlap in therapy is for a period of no less than 4 days.

13. A method according to claim 11 wherein the overlap is 4-5 days.
